(19) United States
(12) Patent Application Publication
(10) Pub. No.: US 2023/0416836 A1
(43) Pub. Date: Dec. 28, 2023

(54) METHOD FOR PREDICTING RESPONSIVENESS TO PLK1 INHIBITION

(71) Applicant: National University of Singapore, Singapore (SG)

(72) Inventors: Anand Devaprasath JEYASEKHARAN, Singapore (SG); Sai Srinivas UPADHYAYULA, Singapore (SG); Mai Phuong HOANG, Singapore (SG); Michal Marek HOPPE, Singapore (SG)

(73) Assignee: National University of Singapore, Singapore (SG)

(21) Appl. No.: 18/252,185

(22) PCT Filed: Nov. 9, 2021
(86) PCT No.: PCT/SG2021/050682

§ 371 Date: May 8, 2023

(30) Foreign Application Priority Data
Nov. 9, 2020 (SG) 10202011116X

(51) Int. Cl. C12Q 1/6886; G01N 33/574

(52) U.S. Cl. CPC C12Q 1/6886; G01N 33/57446; G01N 33/57449; C12Q 2600/106; C12Q 2600/156; G01N 2800/52

(57) ABSTRACT

The present disclosure teaches a method of predicting the responsiveness of a subject towards a Polo-like kinase 1 (PLK1) inhibiting therapy. In one embodiment, there is provided a method for predicting the responsiveness of a subject towards a Polo-like kinase 1 (PLK1) inhibiting therapy, the method comprising determining the level and/or activity of AT-rich interacting domain 1A (ARID1A) in a sample obtained from the subject.

### FIELD OF INVENTION

The invention relates generally to the field of oncology. In particular, the present disclosure teaches a method of predicting the responsiveness of a subject towards a Polo-like kinase 1 (PLK1) inhibiting therapy.

### BACKGROUND

Polo-like kinase 1 (PLK1) is an essential mitotic kinase that plays a role in G2/M cell cycle progression, centrosome maturation, cytokinesis with novel functions described during S phase including DNA replication. PLK1 is overexpressed in many cancers and is associated with poor prognosis although recent evidence suggests a more complex role that is cancer type dependent. Nonetheless, owing to its critical functions in cell survival, it is an attractive target in cancer treatment. Phase II clinical trials with Volasertib; a potent PLK1 inhibitor showed encouraging results as monotherapy, but there are no biomarkers to date that can be used to enrich for responders.

Accordingly, it is generally desirable to overcome or ameliorate one or more of the above mentioned difficulties.

### SUMMARY

Disclosed herein is a method for predicting the responsiveness of a subject towards a Polo-like kinase 1 (PLK1) inhibiting therapy, the method comprising determining the level and/or activity of AT-rich interacting domain 1A (ARID1A) in a sample obtained from the subject.

Disclosed herein is a method for predicting the responsiveness of a subject towards a Polo-like kinase 1 (PLK1) inhibiting therapy, the method comprising determining the mutation status of AT-rich interacting domain 1A (ARID1A) in a sample obtained from the subject.

Disclosed herein is a method for predicting the responsiveness of a subject towards a Polo-like kinase 1 (PLK1) inhibiting therapy, the method comprising determining transcriptomic and/or metabolic changes due to AT-rich interacting domain 1A (ARID1A) mutation(s) or loss in a sample obtained from the subject.

Disclosed herein is a method of treating a subject with a PLK1 inhibiting therapy, the method comprising; a) determining the level and/or activity of AT-rich interacting domain 1A (ARID1A) in a sample obtained from the subject to predict the responsiveness of the subject towards the PLK1 inhibiting therapy; and b) treating a subject found likely to be sensitive towards the PLK1 inhibiting therapy.

Disclosed herein is a method of stratifying a subject as a likely responder or non-responder towards PLK1 inhibiting therapy, the method comprising determining the level and/or activity of AT-rich interacting domain 1A (ARID1A) in a sample obtained from the subject to predict the responsiveness of the subject towards the PLK1 inhibiting therapy, to thereby stratify the subject as a likely responder or non-responder towards the PLK1 inhibiting therapy.

Disclosed herein is a method of predicting the treatment outcome of a subject towards a PLK1 inhibiting therapy, the method comprising determining the level and/or activity of AT-rich interacting domain 1A (ARID1A) in a sample obtained from the subject to predict the responsiveness of the subject towards the PLK1 inhibiting therapy; and thereby predict the treatment outcome of the subject towards the PLK1 inhibiting therapy.

### BRIEF DESCRIPTION OF DRAWINGS

Embodiments of the present invention are hereafter described, by way of non-limiting example only, with reference to the accompanying drawings in which:

FIG. 1: TCGA data analysis showing prevalence of ARID1A mutations in cancers. Histogram representing percentage of cases with ARID1A mutations in different types of cancer (data obtained from TCGA, data accessed on 16 Oct. 2019).

FIG. 2: Generation of ARID1A KO cells in (A) GES1, (B) MCF10A, (C) OVCAR3, (D) Ratios of Z-scores, showing hits from preliminary FDA approved kinase inhibitor screen.

FIG. 3: Validation of the screen confirming synthetic lethality between ARID1A loss and Volasertib. Cell titre blue assay confirming synthetic lethality between ARID1A loss and Volasertib in (A) GES1, (B) MCF10A, (C) Quantitation of colonies from colony formation assay in GES1 ARID1A-WT and KO cells, (D) Representative images of colony formation assay showing synthetic lethality between ARID1A loss and Volasertib in GES1 cells, (E) Cell proliferation assays showing synthetic lethality between ARID1A loss and Volasertib in OVCAR3 cells.

FIG. 4: Anti-tumor activity of Volasertib on OVCAR3-ARID1A KO xenografts (A) Schematic representation of mouse xenograft experiments. Nude (SCID) mice received sub-cutaneous injections of OVCAR3-ARID1A WT or KO cells. Tumors were visible 10 days post injection, after which the animals were treated with Volasertib (15 mg/kg body weight) or vehicle once per week for 4 weeks. Body mass and tumor volumes were recorded twice per week. Mice were sacrificed at the end of 4 weeks of treatment and tumors were isolated and imaged, (B) Body mass measurements of mice measured twice per week, (C) Tumor volume measurements of mice measured twice per week, p<0.05, student's t-test, (D) Images of OVCAR3-ARID1A WT/KO tumors isolated from mice after sacrifice, (E) Dot plot of tumor area from D showing changes in tumor area in OVCAR3-ARID1A KO vs OVCAR3-ARID1A KO+volasertib treatment. Tumor area was calculated from the images in image J using the ruler to set scale and applying free hand tool to measure the tumor area.

FIG. 5: Western blots showing increased cleaved PARP/apoptosis at baseline levels in (A) GES1 cells, (B) OVCAR3 cells, (C) Representative images of mitochondrial depolarization measured in GES1 cells using JC-1 showing increased depolarization in GES1-ARID1A KO cells at baseline level and increased depolarization post Volasertib treatment in a time dependent manner, (D) Quantitation of depolarized mitochondria using JC-1 assay (n=3), (E) Annexin-V staining performed in GES1 ARID1A-WT and KO cells showing higher baseline apoptosis in ARID1A-KO cells with further increase post Volasertib treatment, (F) Quantitation of Annexin-V positive cells in GES1 ARID1A-WT and KO cells showing higher apoptosis in ARID1A KO cells (n=3), Western blots using whole cell lysates from cells treated with Volasertib for the indicated time points showing increased accumulation of cleaved PARP in (G) GES1 cells, (H) OVCAR3 cells. HSC70 was used as loading control.

FIG. 6: RNA sequencing analysis of GES1-ARID1A WT and KO cells (A) Volcano plot showing transcriptomic changes in GES1-ARID1A WT and KO cells, (B) GSEA analysis of enriched pathways in GES1-ARID1A KO cells, (C) Enrichment plot of reactive oxygen pathway in GES1-ARID1A KO cells from GSEA analysis in B, (D) Enrichment plot of oxidative phosphorylation pathway in GES1-ARID1A KO cells from GSEA analysis in B, (E) Volcano plot showing transcriptomic changes in GES1-ARID1A WT and KO cells treated with volasertib, (F) Enrichment plot of oxidative phosphorylation pathway in GES1-ARID1A KO cells from GSEA analysis in (E), (G) Enrichment plot of reactive oxygen pathway in GES1-ARID1A KO cells from GSEA analysis in (E).

FIG. 7: (A) PCA plot of GES1-ARID1A isogenic cell lines with and without volasertib treatment showing differences in metabolic intermediates, as measured by 1D NMR, between ARID1A WT and KO cells, which is exacerbated upon volasertib treatment. (B) Heat map of important metabolites up/down regulated in GES1-ARID1A WT and KO with and without volasertib treatment, as measured by 1D NMR.

FIG. 8: (A) Overview of the principle underlying an ARID1A quantitative IHC assay to predict PLKli sensitivity. (B) Validation of an anti-ARID1A antibody specificity for formalin-fixed samples; using OVCAR-3 ARID1A-expressing and knock-out (KO) formalin-fixed cell blocks. (C) Schematic representation of machine-learning assisted analysis of ARID1A multiplexed fluorescent IHC samples, using EpCAM as an epithelial marker to differentiate epithelial tumor areas from stroma. (D) Examples of unmixed monochrome images of gastric cancer samples with ARID1A-Loss (left) and intact ARID1A (right). ARID1A is retained within the stroma of ARID1A deficient cancers, and serves as an internal control. Quantitative ARID1A_{NE}S Ratio between Tumor/Stromal compartments is indicated above the images. Cell segmentation and tissue segmentation masks were created based on unmixed EpCAM and DAPI nuclear counterstaining images. DAPI—4′,6-diamidino-2-phenylindole. Scale bar is 50 m in all panels.

### DETAILED DESCRIPTION

The present specification teaches a method for predicting the responsiveness of a subject towards a Polo-like kinase 1 (PLK1) inhibiting therapy.

Disclosed herein is a method for predicting the responsiveness of a subject towards a Polo-like kinase 1 (PLK1) inhibiting therapy, the method comprising determining the level and/or activity of AT-rich interacting domain 1A (ARID1A) in a sample obtained from the subject.

Disclosed herein is a method for predicting the responsiveness of a subject towards a Polo-like kinase 1 (PLK1) inhibiting therapy, the method comprising determining the mutation status of AT-rich interacting domain 1A (ARID1A) in a sample obtained from the subject.

Disclosed herein is a method for predicting the responsiveness of a subject towards a Polo-like kinase 1 (PLK1) inhibiting therapy, the method comprising determining transcriptomic and/or metabolic changes due to AT-rich interacting domain 1A (ARID1A) mutation(s) or loss in a sample obtained from the subject.

AT-rich interacting domain 1A (ARID1A) is a key subunit of the SWI/SWF chromatin remolding complex and an important regulator of gene expression. ARID1A acts as a tumor suppressor and is mutated in 10-20% of all cancers. ARID1A mutations are particularly common cancers that are prominent in Asia, for example, 20% in gastric cancer and 40% in ovarian clear cell cancer. These mutations often lead to a loss of protein expression.

Using a high-throughput chemical library screen, the inventors have discovered that ARID1A knockout cells are exquisitely sensitive to PLK1 inhibition. The inventors have therefore identified a method for prediction of PLK1 sensitivity for the stratification of patients for PLK1 inhibitor treatment. Based on this premise the inventors have optimized a high-throughput ARID1A detection assay, which can be applied to patient biopsies to rapidly and inexpensively diagnose patients. The assay employs immunohistochemical (IHC) techniques and machine learning algorithms to accurately detect and quantitate ARID1A expression. It is anticipated that using this assay to accurately quantitate ARID1A expression in patient biopsies will enable effective stratification of patients for inclusion or exclusion into PLK1 inhibitor clinical trials. Additionally, the inventors have created ARID1A knockout (KO) cell lines, which have been used to derive a gene expression signature of ARID1A loss. Such a signature could also be used as a surrogate biomarker for the identification of loss of ARID1A function in tumors, to select patients for PLK1 inhibition.

The term “responsiveness” to therapy may refer to any one or more of: extending survival (including overall survival and progression free survival); resulting in an objective response (including a complete response or a partial response); or improving signs or symptoms of cancer. In some embodiments, responsiveness may refer to improvement of one or more factors according to the published set of RECIST guidelines for determining the status of a tumor in a cancer patient, i.e., responding, stabilizing, or progressing. A responsive subject may refer to a subject whose cancer(s) show improvement, e.g., according to one or more factors based on RECIST criteria. A non-responsive subject may refer to a subject whose cancer(s) do not show improvement, e.g., according to one or more factors based on RECIST criteria.

In one embodiment, a responsive subject refers to a subject who shows a complete response (CR) or a partial response (PR), while a non-responsive subject is one who does not show improvement and has a stable disease (SD) or progressive disease (PD).

The term “predicting the responsiveness” may also refer to determining the likelihood of a subject who is responsive to a cancer therapy.

The method may comprise detecting the level and/or activity of the ARID1A biomarker.

In one embodiment, an increased level and/or activity of ARID1A as compared to a reference indicates a likelihood of resistance towards the PLK1 inhibiting therapy.

In one embodiment, a decreased level and/or activity of ARID1A as compared to a reference indicates a likelihood of sensitivity towards the PLK1 inhibiting therapy.

In one embodiment, there is provided a method for predicting the responsiveness of a subject towards a Polo-like kinase 1 (PLK1) inhibiting therapy, the method comprising determining the level and/or activity of AT-rich interacting domain 1A (ARID1A) in a sample obtained from the subject, wherein an increased level and/or activity of ARID1A as compared to a reference indicates a likelihood of resistance towards the PLK1 inhibiting therapy.

In one embodiment, there is provided a method for predicting the responsiveness of a subject towards a Polo-like kinase 1 (PLK1) inhibiting therapy, the method comprising determining the level and/or activity of AT-rich interacting domain 1A (ARID1A) in a sample obtained from the subject, wherein a decreased level and/or activity of ARID1A as compared to a reference indicates a likelihood of sensitivity towards the PLK1 inhibiting therapy.

In one embodiment, the decreased level and/or activity of ARID1A as compared to a reference is due to a loss-of-function mutation or deletion, epigenetic silencing or a loss of mRNA stability.

The term “biomarker” as used herein refers to an indicator, e.g., predictive, diagnostic, and/or prognostic, which can be detected in a sample. The biomarker may serve as an indicator of a particular subtype of a disease or disorder (e.g., cancer), characterized by certain, molecular, pathological, histological, and/or clinical features, and/or may serve as an indicator of a particular cell type or state (e.g., epithelial, mesenchymal etc.) and/or or response to therapy. Biomarkers include, but are not limited to, polynucleotides (e.g., DNA, and/or RNA), polynucleotide copy number alterations (e.g., DNA copy numbers), polypeptides, polypeptide and polynucleotide modifications (e.g., posttranslational modifications), carbohydrates, and/or glycolipid-based molecular markers. A biomarker may be present in a sample obtained from a subject before the onset of a physiological or pathophysiological state (e.g., primary cancer, metastatic cancer, etc.), including a symptom, thereof (e.g., response to therapy). Thus, the presence of the biomarker in a sample obtained from the subject can be indicative of an increased risk that the subject will develop the physiological or pathophysiological state or symptom thereof. Alternatively, or in addition, the biomarker may be normally expressed in an individual, but its expression may change i.e., it is increased (upregulated; over-expressed) or decreased (downregulated; under-expressed) before the onset of a physiological or pathophysiological state, including a symptom thereof. Thus, a change in the level of the biomarker may be indicative of an increased risk that the subject will develop the physiological or pathophysiological state or symptom thereof. Alternatively, or in addition, a change in the level of a biomarker may reflect a change in a particular physiological or pathophysiological state, or symptom thereof, in a subject, thereby allowing the nature (e.g., severity) of the physiological or pathophysiological state, or symptom thereof, to be tracked over a period of time. This approach may be useful in, for example, monitoring a treatment regimen for the purpose of assessing its effectiveness (or otherwise) in a subject. As herein described, reference to the level of a biomarker includes the concentration of a biomarker, or the level of expression of a biomarker, or the activity of the biomarker.

In one embodiment, the biomarker is ARID1A. In one embodiment, the biomarker is a gene or protein that is regulated by ARID1A.

The “amount” or “level” of a biomarker is a detectable level or amount in a sample. These can be measured by methods known to one skilled in the art and also disclosed herein. These terms encompass a quantitative amount or level (e.g., weight or moles), a semi-quantitative amount or level, a relative amount or level (e.g., weight % or mole % within class), a concentration, and the like. Thus, these terms encompass absolute or relative amounts or levels or concentrations of a biomarker in a sample. The expression level or amount of biomarker assessed can be used to determine the response to treatment.

The term “detection” includes any means of detecting, including direct and indirect detection.

The term “expression” with respect to a gene sequence refers to transcription of the gene to produce a RNA transcript (e.g., mRNA, antisense RNA, siRNA, shRNA, miRNA, etc.) and, as appropriate, translation of a resulting mRNA transcript to a protein. Thus, as will be clear from the context, expression of a coding sequence results from transcription and translation of the coding sequence. Conversely, expression of a non-coding sequence results from the transcription of the non-coding sequence.

The terms “level of expression” or “expression level” in general are used interchangeably and generally refer to the amount of a biomarker in a sample. “Expression” generally refers to the process by which information (e.g., gene-encoded and/or epigenetic) is converted into the structures present and operating in the cell. Therefore, as used herein, “expression” may refer to transcription into a polynucleotide, translation into a polypeptide, or even polynucleotide and/or polypeptide modifications (e.g., posttranslational modification of a polypeptide). Fragments of the transcribed polynucleotide, the translated polypeptide, or polynucleotide and/or polypeptide modifications (e.g., post-translational modification of a polypeptide) shall also be regarded as expressed whether they originate from a transcript generated by alternative splicing or a degraded transcript, or from a post-translational processing of the polypeptide, e.g., by proteolysis. “Expressed genes” include those that are transcribed into a polynucleotide as mRNA and then translated into a polypeptide, and also those that are transcribed into RNA but not translated into a polypeptide (e.g., transfer and ribosomal RNAs). Thus, “elevated expression”, “elevated expression levels”, or “elevated levels” refers to an increased expression or increased levels of a biomarker in a cell or individual relative to a control, such as a cell or cells that are responding or not responding to a therapy, or an individual or individuals who are responding or not responding to a therapy, or an internal control (e.g., housekeeping biomarker). “Reduced expression”, “reduced expression levels”, or “reduced levels” refers to a decreased expression or decreased levels of a biomarker in an individual relative to a control, such as a cell or cells that are responding or not responding to a therapy, or an individual or individuals who are responding or not responding to a therapy, an internal control (e.g., housekeeping biomarker). In some embodiments, reduced expression is little or no expression

As used herein, the term “increase” or “increased’ with reference to the level and/or activity of a biomarker refers to a statistically significant and measurable increase in the biomarker compared to the level of another biomarker or to a control level. The increase is preferably an increase of at least about 10%, or an increase of at least about 20%, or an increase of at least about 30%, or an increase of at least about 40%, or an increase of at least about 50%.

As used herein, the term “higher” with reference to a biomarker refers to a statistically significant and measurable difference in the level of a biomarker compared to the level of another biomarker or to a control level where the biomarker is greater than the level of the other biomarker or the control level. The difference is preferably at least about 10%, or at least about 20%, or of at least about 30%, or of at least about 40%, or at least about 50%.

As used herein, the term “reduce” or “reduced” with reference to a biomarker refers to a statistically significant and measurable reduction in the biomarker compared to the level of another biomarker or to a control level. The reduction is preferably a reduction of at least about 10%, or a reduction of at least about 20%, or a reduction of at least about 30%, or a reduction of at least about 40%, or a reduction of at least about 50%.

As used herein, the term “lower” with reference to a biomarker refers to a statistically significant and measurable difference in the level of a biomarker compared to the level of another biomarker or biomarker complex or to a control level where the biomarker is less than the level of the other biomarker or the control level. The difference is preferably at least about 10%, or at least about 20%, or of at least about 30%, or of at least about 40%, or at least about 50%.

The term “sample” as used herein includes any biological specimen that may be extracted, untreated, treated, diluted or concentrated from a subject. A sample includes within its scope a collection of similar fluids, cells, or tissues (e.g., surgically resected tumor tissue, biopsies, including fine needle aspiration), isolated from a subject, as well as fluids, cells, or tissues present within a subject. In some embodiments the sample is a biological fluid. Biological fluids are typically liquids at physiological temperatures and may include naturally occurring fluids present in, withdrawn from, expressed or otherwise extracted from a subject or biological source. Certain biological fluids derive from particular tissues, organs or localized regions and certain other biological fluids may be more globally or systemically situated in a subject or biological source. Examples of biological fluids include blood, serum and serosal fluids, plasma, lymph, urine, saliva, cystic fluid, tear drops, feces, sputum, mucosal secretions of the secretory tissues and organs, vaginal secretions, ascites fluids such as those associated with non-solid tumors, fluids of the pleural, pericardial, peritoneal, abdominal and other body cavities, fluids collected by bronchial lavage and the like. Biological fluids may also include liquid solutions contacted with a subject or biological source, for example, cell and organ culture medium including cell or organ conditioned medium, lavage fluids and the like. The term “sample” as used herein encompasses materials removed from a subject or materials present in a subject.

A “reference sample”, “reference cell”, “reference tissue”, “control sample”, “control cell”, or “control tissue”, as used herein, refers to a sample, cell, tissue, standard, or level that is used for comparison purposes (i.e. as a reference). In one embodiment, a reference sample, reference cell, reference tissue, control sample, control cell, or control tissue is obtained from a healthy and/or non-diseased part of the body (e.g., tissue or cells) of the same subject or individual. For example, healthy and/or non-diseased cells or tissue adjacent to the diseased cells or tissue. In another embodiment, a reference sample is obtained from an untreated tissue and/or cell of the body of the same subject or individual. In yet another embodiment, a reference sample, reference cell, reference tissue, control sample, control cell, or control tissue is obtained from a healthy and/or non-diseased part of the body (e.g., tissues or cells) of an individual who is not the subject or individual. In even another embodiment, a reference sample, reference cell, reference tissue, control sample, control cell, or control tissue is obtained from an untreated tissue and/or cell of the body of an individual who is not the subject or individual.

In certain embodiments, the samples are normalized for both differences in the amount of the biomarker assayed and variability in the quality of the samples used, and variability between assay runs. Such normalization may be accomplished by detecting and incorporating the expression of certain normalizing biomarkers, including expression products of well-known housekeeping genes. Alternatively, normalization can be based on the mean or median signal of all of the assayed genes or a large subset thereof (global normalization approach). On a gene-by-gene basis, measured normalized amount of a subject tumor mRNA or protein is compared to the amount found in a reference set. Normalized expression levels for each mRNA or protein per tested tumor per subject can be expressed as a percentage of the expression level measured in the reference set. The presence and/or expression level/amount measured in a particular subject sample to be analyzed will fall at some percentile within this range, which can be determined by methods well known in the art.

In some embodiments, the sample is a clinical sample. In some embodiments, the sample is obtained from a primary or metastatic tumor. Tissue biopsy is often used to obtain a representative piece of tumor tissue. Alternatively, tumor cells can be obtained indirectly in the form of tissues or fluids that are known or thought to contain the tumor cells of interest. Genes or gene products can be detected from cancer or tumor tissue or from other body samples such as urine, sputum, serum or plasma. The same techniques discussed above for detection of target genes or gene products in cancerous samples can be applied to other body samples. Cancer cells may be sloughed off from cancer lesions and appear in such body samples. By screening such body samples, a simple early diagnosis can be achieved for these cancers. In addition, the progress of therapy can be monitored more easily by testing such body samples for target genes or gene products.

In certain embodiments, a reference sample, reference cell, reference tissue, control sample, control cell, or control tissue is a single sample or combined multiple samples from the same subject or individual that are obtained at one or more different time points than when the test sample is obtained. For example, a reference sample, reference cell, reference tissue, control sample, control cell, or control tissue is obtained at an earlier time point from the same subject or individual than when the test sample is obtained. Such reference sample, reference cell, reference tissue, control sample, control cell, or control tissue may be useful if the reference sample is obtained during initial diagnosis of cancer and the test sample is later obtained when the cancer becomes metastatic.

In certain embodiments, a reference sample, reference cell, reference tissue, control sample, control cell, or control tissue is a combination of multiple samples from one or more healthy individuals who are not the subject or individual. In certain embodiments, a reference sample, reference cell, reference tissue, control sample, control cell, or control tissue is a combination of multiple samples from one or more individuals with a disease or disorder (e.g., cancer) who are not the subject or individual. In certain embodiments, a reference sample, reference cell, reference tissue, control sample, control cell, or control tissue is pooled RNA samples from normal tissues or pooled plasma or serum samples from one or more individuals who are not the subject or individual. In certain embodiments, a reference sample, reference cell, reference tissue, control sample, control cell, or control tissue is pooled RNA samples from tumor tissues or pooled plasma or serum samples from one or more individuals with a disease or disorder (e.g., cancer) who are not the subject or individual.

In some embodiments, the sample is a tissue sample from the individual. In some embodiments, the tissue sample is a tumor tissue sample (e.g., biopsy tissue). In some embodiments, the tissue sample is lung tissue. In some embodiments, the tissue sample is renal tissue. In some embodiments, the tissue sample is skin tissue. In some embodiments, the tissue sample is pancreatic tissue. In some embodiments, the tissue sample is gastric tissue. In some embodiments, the tissue sample is bladder tissue. In some embodiments, the tissue sample is esophageal tissue. In some embodiments, the tissue sample is mesothelial tissue. In some embodiments, the tissue sample is breast tissue. In some embodiments, the tissue sample is thyroid tissue. In some embodiments, the tissue sample is colorectal tissue. In some embodiments, the tissue sample is head and neck tissue. In some embodiments, the tissue sample is osteosarcoma tissue. In some embodiments, the tissue sample is prostate tissue. In some embodiments, the tissue sample is ovarian tissue, HCC (liver), blood cells, lymph nodes, and/or bone/bone marrow tissue. In some embodiments, the tissue sample is colon tissue. In some embodiments, the tissue sample is endometrial tissue. In some embodiments, the tissue sample is brain tissue (e.g., glioblastoma, neuroblastoma, and so forth).

In some embodiments, a tumor tissue sample (the term “tumor sample” is used interchangeably herein) may encompass part or all of the tumor area occupied by tumor cells. In some embodiments, a tumor or tumor sample may further encompass tumor area occupied by tumor associated intratumoral cells and/or tumor associated stroma (e.g., contiguous peri-tumoral desmoplastic stroma). Tumor associated intratumoral cells and/or tumor associated stroma may include areas of immune infiltrates immediately adjacent to and/or contiguous with the main tumor mass.

In some embodiments, tumor cell staining is expressed as the percent of all tumor cells showing staining (e.g., membranous, cytoplasmic or nuclear staining) of any intensity. Infiltrating immune cell staining may be expressed as the percent of the total tumor area occupied by immune cells that show staining of any intensity. The total tumor area encompasses the malignant cells as well as tumor-associated stroma, including areas of immune infiltrates immediately adjacent to and contiguous with the main tumor mass. In addition, infiltrating immune cell staining may be expressed as the percent of all tumor infiltrating immune cells.

In some embodiments, the tumor is a malignant cancerous tumor (i.e., cancer). In some embodiments, the tumor and/or cancer is a solid tumor or a non-solid or soft tissue tumor. Examples of soft tissue tumors include leukemia (e.g., chronic myelogenous leukemia, acute myelogenous leukemia, adult acute lymphoblastic leukemia, acute myelogenous leukemia, mature B-cell acute lymphoblastic leukemia, chronic lymphocytic leukemia, prolymphocytic leukemia, or hairy cell leukemia) or lymphoma (e.g., non-Hodgkin's lymphoma, cutaneous T-cell lymphoma, or Hodgkin's disease). A solid tumor includes any cancer of body tissues other than blood, bone marrow, or the lymphatic system. Solid tumors can be further divided into those of epithelial cell origin and those of non-epithelial cell origin. Examples of epithelial cell solid tumors include tumors of the gastrointestinal tract, colon, colorectal (e.g., basaloid colorectal carcinoma), breast, prostate, lung, kidney, liver, pancreas, ovary (e.g., endometrioid ovarian carcinoma), head and neck, oral cavity, stomach, duodenum, small intestine, large intestine, anus, gall bladder, labium, nasopharynx, skin, uterus, male genital organ, urinary organs (e.g., urothelium carcinoma, dysplastic urothelium carcinoma, transitional cell carcinoma), bladder, and skin. Solid tumors of non-epithelial origin include sarcomas, brain tumors, and bone tumors. In some embodiments, the cancer is second-line or third-line locally advanced or metastatic non-small cell lung cancer. In some embodiments, the cancer is adenocarcinoma. In some embodiments, the cancer is squamous cell carcinoma. In some embodiments, the cancer is non-small cell lung cancer (NSCLC), glioblastoma, neuroblastoma, melanoma, breast carcinoma (e.g. triple-negative breast cancer), gastric cancer, colorectal cancer (CRC), or hepatocellular carcinoma. In some embodiments, the cancer is a primary tumor. In some embodiments, the cancer is a metastatic tumor at a second site derived from any of the above types of cancer.

In one embodiment, the subject is suffering from cancer and the sample that is obtained from the subject comprises cancer cells. Cancer cells for the practice of the present invention can be obtained from any suitable cancer-cell containing patient samples, illustrative examples of which include tumor biopsies, circulating tumor cells, primary cell cultures or cell lines derived from tumors or exhibiting tumor-like properties, as well as preserved tumor samples, such as formalin-fixed, paraffin-embedded tumor samples or frozen tumor samples. In some embodiments, the sample is obtained prior to treatment with a therapy. In other embodiments, the sample is obtained after treatment with a therapy. In some embodiments, the sample comprises a tissue sample, which can be formalin fixed and paraffin embedded, archival, fresh or frozen. In some embodiments, the sample is whole blood. In some embodiments, the whole blood comprises circulating tumor cells.

Presence and/or levels/amount of a biomarker can be determined qualitatively and/or quantitatively based on any suitable criterion known in the art, including but not limited to proteins and protein fragments. In certain embodiments, presence and/or expression levels/amount of a biomarker in a first sample is increased or elevated as compared to presence/absence and/or expression levels/amount in a second sample (e.g., before treatment with a therapy). In certain embodiments, presence/absence and/or levels/amount of a biomarker in a first sample is decreased or reduced as compared to presence and/or levels/amount in a second sample. In certain embodiments, the second sample is a reference sample, reference cell, reference tissue, control sample, control cell, or control tissue. Additional disclosures for determining presence/absence and/or levels/amount of a gene are described herein.

Presence and/or level/amount of various biomarkers in a sample can be analyzed by a number of methodologies, many of which are known in the art and understood by the skilled artisan, including, but not limited to, immunohistochemistry (“IHC”), Western blot analysis, immunoprecipitation, molecular binding assays, ELISA, ELIFA, fluorescence activated cell sorting (“FACS”), MassARRAY, proteomics, quantitative blood based assays (as for example Serum ELISA), biochemical enzymatic activity assays, in situ hybridization, Southern analysis, Northern analysis, whole genome sequencing, polymerase chain reaction (“PCR”) including quantitative real time PCR (“qRT-PCR”) and other amplification type detection methods, such as, for example, branched DNA, SISBA, TMA and the like), RNA-Seq, FISH, microarray analysis, gene expression profiling, and/or serial analysis of gene expression (“SAGE”), as well as any one of the wide variety of assays that can be performed by protein, gene, and/or tissue array analysis. Typical protocols for evaluating the status of genes and gene products are found, for example in Ausubel et al., eds., 1995, Current Protocols In Molecular Biology, Units 2 (Northern Blotting), 4 (Southern Blotting), 15 (Immunoblotting) and 18 (PCR Analysis). Multiplexed immunoassays such as those available from Rules Based Medicine or Meso Scale Discovery (“MSD”) may also be used.

In some embodiments, presence and/or level/amount of a biomarker is determined using a method comprising: (a) performing gene expression profiling, PCR (such as RT-PCR or qRT-PCR), RNA-seq, microarray analysis, SAGE, MassARRAY technique, or FISH on a sample (such as a subject cancer sample); and b) determining presence and/or expression level/amount of a biomarker in the sample. In some embodiments, the microarray method comprises the use of a microarray chip having one or more nucleic acid molecules that can hybridize under stringent conditions to a nucleic acid molecule encoding a gene mentioned above or having one or more polypeptides (such as peptides or antibodies) that can bind to one or more of the proteins encoded by the genes mentioned above. In one embodiment, the PCR method is qRT-PCR. In one embodiment, the PCR method is multiplex-PCR. In some embodiments, gene expression is measured by microarray. In some embodiments, gene expression is measured by qRT-PCR. In some embodiments, expression is measured by multiplex-PCR.

Methods for the evaluation of mRNAs in cells are well known and include, for example, hybridization assays using complementary DNA probes (such as in situ hybridization using labeled riboprobes specific for the one or more genes, Northern blot and related techniques) and various nucleic acid amplification assays (such as RT-PCR using complementary primers specific for one or more of the genes, and other amplification type detection methods, such as, for example, branched DNA, SISBA, TMA and the like).

The term “label” when used herein refers to a detectable compound or composition. The label is typically conjugated or fused directly or indirectly to a reagent, such as a polynucleotide probe or an antibody, and facilitates detection of the reagent to which it is conjugated or fused. The label may itself be detectable (e.g., radioisotope labels or fluorescent labels) or, in the case of an enzymatic label, may catalyze chemical alteration of a substrate compound or composition which results in a detectable product.

Samples from mammals can be conveniently assayed for mRNAs using Northern, dot blot or PCR analysis. In addition, such methods can include one or more steps that allow one to determine the levels of target mRNA in a biological sample (e.g., by simultaneously examining the levels a comparative control mRNA sequence of a “housekeeping” gene such as an actin family member). Optionally, the sequence of the amplified target cDNA can be determined.

Optional methods include protocols which examine or detect mRNAs, such as target mRNAs, in a tissue or cell sample by microarray technologies. Using nucleic acid microarrays, test and control mRNA samples from test and control tissue samples are reverse transcribed and labeled to generate cDNA probes. The probes are then hybridized to an array of nucleic acids immobilized on a solid support. The array is configured such that the sequence and position of each member of the array is known. For example, a selection of genes whose expression correlates with increased or reduced clinical benefit of a therapy may be arrayed on a solid support. Hybridization of a labeled probe with a particular array member indicates that the sample from which the probe was derived expresses that gene.

In preferred embodiments, presence and/or level/amount is measured by observing protein levels. In certain embodiments, the method comprises contacting the biological sample with an antibody to a biomarker under conditions permissive for binding of the biomarker(s), and detecting whether a complex is formed between the antibody or antibodies and the biomarker(s). Such method may be an in vitro or in vivo method. In some embodiments, one or more anti-biomarker antibodies are used to select subjects eligible for a therapy.

In certain embodiments, the presence and/or expression level/amount of biomarker proteins in a sample is examined using IHC and staining protocols. IHC staining of tissue sections has been shown to be a reliable method of determining or detecting presence of proteins in a sample. In some embodiments, the level of a biomarker in a sample from an individual is an elevated level and, in further embodiments, is determined using IHC. In some embodiments, the level of a biomarker in a sample from an individual is a reduced level and, in further embodiments, is determined using IHC. In one embodiment, the level of biomarker is determined using a method comprising: (a) performing IHC analysis of a sample (such as a subject cancer sample) with an antibody; and b) determining the level of a biomarker in the sample. In some embodiments, IHC staining intensity is determined relative to a reference. In some embodiments, the reference is a reference value. In some embodiments, the reference is a reference sample (e.g., control cell line staining sample or tissue sample from non-cancerous patient).

In some embodiments, the expression of a biomarker is evaluated on a tumor or tumor sample. As used herein, a tumor or tumor sample may encompass part or all of the tumor area occupied by tumor cells. In some embodiments, a tumor or tumor sample may further encompass tumor area occupied by tumor associated intratumoral cells and/or tumor associated stroma (e.g., contiguous peri-tumoral desmoplastic stroma). Tumor associated intratumoral cells and/or tumor associated stroma may include areas of immune infiltrates (e.g., tumor infiltrating immune cells as described herein) immediately adjacent to and/or contiguous with the main tumor mass. In some embodiments, biomarker expression is evaluated on tumor cells.

In alternative methods, the sample may be contacted with an antibody specific for said biomarker under conditions sufficient for an antibody-biomarker complex to form, and then detecting said complex. The presence of the biomarker may be detected in a number of ways, such as by Western blotting and ELISA procedures for assaying a wide variety of tissues and samples, including plasma or serum. A wide range of immunoassay techniques using such an assay format are available, see, e.g., U.S. Pat. Nos. 4,016,043, 4,424,279 and 4,018,653. These include both single-site and two-site or “sandwich” assays of the non-competitive types, as well as in the traditional competitive binding assays. These assays also include direct binding of a labeled antibody to a target biomarker.

In one embodiment, the method further comprises detecting a tumor biomarker in the one or more cancer cells in the sample. The tumor biomarker may be an epithelial biomarker to distinguish tumor cells from stromal cells in an epithelial cancer (for tumor/stroma differentiation). In one embodiment, the epithelial biomarker is Epcam.

In one embodiment, the method comprising determining the mutation status of AT-rich interacting domain 1A (ARID1A) in a sample obtained from the subject. This may be done using techniques such as sequencing. This may lead to a reduction of the level and/or activity of ARID1A.

In one embodiment, the method comprising determining transcriptomic and/or metabolic changes due to AT-rich interacting domain 1A (ARID1A) mutation(s) or loss in a sample obtained from the subject. For example, the transcriptomic changes may be determined by RNA sequencing analysis while metabolic changes may be determined using NMR spectroscopy (e.g. 1D NMR).

The present invention discloses the detection and quantitation of ARID1A using antigen-binding molecules that bind specifically to the biomarkers. Such antigen-binding molecules may be produced by standard antibody production methods, such as anti-peptide antibody methods. For example, an antibody that binds specifically to ARID1A can be produced by immunizing an animal with a peptide antigen derived from ARID1A.

As used herein, the term “antibody” includes, but is not limited to, synthetic antibodies, monoclonal antibodies, recombinantly produced antibodies, multispecific antibodies (including bi-specific antibodies), human antibodies, humanized antibodies, chimeric antibodies, single-chain Fvs (scFv), Fab fragments, F(ab′) fragments, disulfide-linked Fvs (sdFv) (including bi-specific sdFvs), and anti-idiotypic (anti-Id) antibodies, and epitope-binding fragments of any of the above. The antibodies provided herein may be monospecific, bispecific, trispecific or of greater multi-specificity.

Polyclonal antibodies of the invention may be produced according to standard techniques by immunizing a suitable animal (e.g., rabbit, goat, etc.) with a peptide antigen derived from ARID1A, and separating the polyclonal antibodies from the immune serum, in accordance with standard procedures.

Peptide antigens suitable for producing antibodies of the invention may be designed, constructed and employed in accordance with well-known techniques. See, e.g., ANTIBODIES: A LABORATORY MANUAL, Chapter 5, p. 75-76, Harlow & Lane Eds., Cold Spring Harbor Laboratory (1988); Czernik, Methods In Enzymology, 201: 264-283 (1991); Merrifield, J. Am. Chem. Soc. 85:21-49 (1962)).

Monoclonal antibodies of the invention may be produced in a hybridoma cell line according to the well-known technique of Kohler and Milstein. See Nature 265:495-97 (1975); Kohler and Milstein, Eur. J. Immunol. 6: 511 (1976); see also, CURRENT PROTOCOLS IN MOLECULAR BIOLOGY, Ausubel et al. Eds. (1989). Monoclonal antibodies so produced are highly specific, and improve the selectivity and specificity of diagnostic assay methods provided by the invention. For example, a solution containing the appropriate antigen may be injected into a mouse or other species and, after a sufficient time (in keeping with conventional techniques), the animal is sacrificed and spleen cells obtained. The spleen cells are then immortalized by fusing them with myeloma cells, typically in the presence of polyethylene glycol, to produce hybridoma cells. Rabbit fusion hybridomas, for example, may be produced as described in U.S. Pat. No. 5,675,063, C. Knight, Issued Oct. 7, 1997. The hybridoma cells are then grown in a suitable selection media, such as hypoxanthine-aminopterin-thymidine (HAT), and the supernatant screened for monoclonal antibodies having the desired specificity, as described below. The secreted antibody may be recovered from tissue culture supernatant by conventional methods such as precipitation, ion exchange or affinity chromatography, or the like.

Monoclonal Fab fragments may also be produced in Escherichia coli by recombinant techniques known to those skilled in the art. See, e.g., W. Huse, Science 246:1275-81 (1989); Mullinax et al., Proc. Nat'l Acad. Sci. 87: 8095 (1990). If monoclonal antibodies of one isotype are preferred for a particular application, particular isotypes can be prepared directly, by selecting from the initial fusion, or prepared secondarily, from a parental hybridoma secreting a monoclonal antibody of different isotype by using the sib selection technique to isolate class-switch variants (Steplewski, et al., Proc. Nat'l. Acad. Sci., 82: 8653 (1985); Spira et al., J. Immunol. Methods, 74: 307 (1984)).

Included within the scope of the present invention are equivalent non-antibody molecules, such as antigen-binding fragments. See, e.g., Neuberger et al., Nature 312: 604 (1984). Such equivalent non-antibody reagents may be suitably employed in the methods of the invention further described below.

Antigen-binding molecules contemplated by the invention may be any type of antibody including immunoglobulins, including IgG, IgM, IgA, IgD, and IgE, and antigen-binding fragments thereof. The antibodies may be monoclonal or polyclonal and may be of any species of origin, including (for example) mouse, rat, rabbit, horse, or human, or may be chimeric antibodies. See, e.g., M. Walker et al., Molec. Immunol. 26: 403-11 (1989); Morrision et al., Proc. Nat'l. Acad. Sci. 81: 6851 (1984); Neuberger et al., Nature 312:604 (1984)). The antibodies may be recombinant monoclonal antibodies produced according to the methods disclosed in U.S. Pat. No. 4,474,893 (Reading) or U.S. Pat. No. 4,816,567 (Cabilly et al.) The antibodies may also be chemically constructed by specific antibodies made according to the method disclosed in U.S. Pat. No. 4,676,980 (Segel et al.).

The invention also provides immortalized cell lines that produce an antibody of the invention. For example, hybridoma clones, constructed as described above, that produce monoclonal antibodies to ARID1A disclosed herein are also provided. Similarly, the invention includes recombinant cells producing an antibody of the invention, which cells may be constructed by well-known techniques; for example the antigen combining site of the monoclonal antibody can be cloned by PCR and single-chain antibodies produced as phage-displayed recombinant antibodies or soluble antibodies in E. coli (see, e.g., ANTIBODY ENGINEERING PROTOCOLS, 1995, Humana Press, Sudhir Paul editor.)

Antigen-binding molecules may be further characterized via IHC staining using normal and diseased cells or tissues. IHC may be carried out according to well-known techniques. See, e.g., ANTIBODIES: A LABORATORY MANUAL, Chapter 10, Harlow & Lane Eds., Cold Spring Harbor Laboratory (1988). Briefly, paraffin-embedded tissue (e.g., tumor tissue) is prepared for immunohistochemical staining by deparaffinizing tissue sections with xylene followed by ethanol; hydrating in water then PBS; unmasking antigen by heating slide in sodium citrate buffer; incubating sections in hydrogen peroxide; blocking in blocking solution; incubating slide in primary antibody and secondary antibody; and finally detecting using ABC avidin/biotin method according to manufacturer's instructions.

Antigen-binding molecules may be further characterized by flow cytometry carried out according to standard methods. See Chow et al., Cytometry (Communications in Clinical Cytometry) 46: 7205-238 (2001). Briefly and by way of example, the following protocol for cytometric analysis may be employed: samples may be centrifuged on Ficoll gradients to remove erythrocytes, and cells may then be fixed with 2% paraformaldehyde for 10 minutes at 37° C. followed by permeabilization in 90% methanol for 30 minutes on ice. Cells may then be stained with the primary antigen-binding molecule of the invention, washed and labeled with a fluorescent-labeled secondary antibody. Additional fluorochrome-conjugated biomarker antibodies may also be added at this time to aid in the identification of the epithelial status of the cells. The cells may then be analyzed on a flow cytometer (e.g. a Beckman Coulter FC500) according to the specific protocols of the instrument used.

Antigen-binding molecules may be advantageously conjugated to fluorescent dyes (e.g., Alexa Fluor 488) for use in multi-parametric analyses along with other biomarker antibodies.

The term “PLK1 inhibiting therapy” may comprise any active agent that reduces the accumulation, function or stability of PLK1; or decrease expression of PLK1 gene, and such agents include without limitation, small molecules and macromolecules such as nucleic acids, peptide, polypeptides, peptidomimetics, carbohydrates, polysaccharides, lipopolysaccharides, lipids or other organic (carbon containing) or inorganic molecules.

The terms “polynucleotide,” “genetic material,” “genetic forms,” “nucleic acids” and “nucleotide sequence” include RNA, cDNA, genomic DNA, synthetic forms and mixed polymers, both sense and antisense strands, and may be chemically or biochemically modified or may contain non-natural or derivatized nucleotide bases, as will be readily appreciated by those skilled in the art.

The terms “polypeptide,” “proteinaceous molecule,” “peptide” and “protein” are used interchangeably herein to refer to a polymer of amino acid residues and to variants and synthetic analogues of the same. Thus, these terms apply to amino acid polymers in which one or more amino acid residues is a synthetic non-naturally-occurring amino acid, such as a chemical analogue of a corresponding naturally-occurring amino acid, as well as to naturally-occurring amino acid polymers. These terms do not exclude modifications, for example, glycosylations, acetylations, phosphorylations and the like. Soluble forms of the subject proteinaceous molecules are particularly useful. Included within the definition are, for example, polypeptides containing one or more analogs of an amino acid including, for example, unnatural amino acids or polypeptides with substituted linkages.

As used herein a “small molecule” refers to a composition that has a molecular weight of less than 3 kilodaltons (kDa), and typically less than 1.5 kilodaltons, and suitably less than about 1 kilodalton. Small molecules may be nucleic acids, peptides, polypeptides, peptidomimetics, carbohydrates, lipids or other organic (carbon-containing) or inorganic molecules. As those skilled in the art will appreciate, based on the present description, extensive libraries of chemical and/or biological mixtures, often fungal, bacterial, or algal extracts, may be screened with any of the assays of the invention to identify compounds that modulate a bioactivity. A “small organic molecule” is an organic compound (or organic compound complexed with an inorganic compound (e.g., metal)) that has a molecular weight of less than 3 kilodaltons, less than 1.5 kilodaltons, or even less than about 1 kDa.

The PLK1 inhibiting therapy may comprise a PLK1 inhibitor. The PLK1 inhibitor may be Aristolactam AIIIa, Scytonemin, Wortmannin, Rigosertib (ON01910.Na), BI 2536, Volasertib (BI 6727), GSK461364A, HMN-176, SBE13, ZK-thiazolidinone (TAL), Compound 36, Compound 15, Compound 38, Onvansertib (NMS-P937), LFM-A13, RO3280, TAK-960, MQSpTPL (SEQ ID NO: 1) or MAGPMQSpTPLNGAKK (SEQ ID NO: 2), LLCSpTPNG (SEQ ID NO: 3), PLHSpT (SEQ ID NO: 4), or TKM-080301.

In one embodiment, the PLK1 inhibiting therapy is Volasertib (B1I6727), Rigosertib (ON01910.Na), Onvansertib (NMS-P937) or B12536.

In some embodiments, the PLK1 inhibitor is an antagonistic nucleic acid molecule that functions to inhibit the transcription or translation of PLK1 transcripts.

Illustrative antagonist nucleic acid molecules include antisense molecules, aptamers, ribozymes and triplex forming molecules, RNAi and external guide sequences. The nucleic acid molecules can act as effectors, inhibitors, modulators, and stimulators of a specific activity possessed by a target molecule, or the functional nucleic acid molecules can possess a de novo activity independent of any other molecules.

Antagonist nucleic acid molecules can interact with any macromolecule, such as DNA, RNA, polypeptides, or carbohydrate chains. Thus, antagonist nucleic acid molecules can interact with PLK1 mRNA or the genomic DNA of PLK1 or they can interact with a PLK1 polypeptide. Often antagonist nucleic acid molecules are designed to interact with other nucleic acids based on sequence homology between the target molecule and the antagonist nucleic acid molecule. In other situations, the specific recognition between the antagonist nucleic acid molecule and the target molecule is not based on sequence homology between the antagonist nucleic acid molecule and the target molecule, but rather is based on the formation of tertiary structure that allows specific recognition to take place.

In one embodiment, the PLK1 inhibiting therapy is a therapy that comprises a PLK1 inhibitor and another therapeutic agent. For example, the PLK1 inhibiting therapy may comprise a PLK1 inhibitor with Vincristine, Vinorelbine, Eribulin, Paclitaxel, Doxorubicin or Etoposide.

In one embodiment, the subject is suffering from cancer.

The terms “cancer” and “cancerous” refer to or describe the physiological condition in mammals that is typically characterized in part by unregulated cell growth. As used herein, the term “cancer” refers to non-metastatic and metastatic cancers, including early stage and late stage cancers. By “non-metastatic” is meant a cancer that remains at the primary site and has not penetrated into the lymphatic or blood vessel system or to tissues other than the primary site. The term “metastatic cancer” refers to cancer that has spread or is capable of spreading from one part of the body to another. Generally, a non-metastatic cancer is any cancer that is a Stage 0, I, or II cancer, and occasionally a Stage III cancer. A metastatic cancer, on the other hand, is usually a stage IV cancer.

The term “cancer” includes but is not limited to, breast cancer, large intestinal cancer, lung cancer, small cell lung cancer, gastric (stomach) cancer, liver cancer, blood cancer, bone cancer, pancreatic cancer, skin cancer, head and/or neck cancer, cutaneous or intraocular melanoma, uterine sarcoma, ovarian cancer, rectal or colorectal cancer, anal cancer, colon cancer, fallopian tube carcinoma, endometrial carcinoma, cervical cancer, vulval cancer, squamous cell carcinoma, vaginal carcinoma, Hodgkin's disease, non-Hodgkin's lymphoma, esophageal cancer, small intestine cancer, endocrine cancer, thyroid cancer, parathyroid cancer, adrenal cancer, soft tissue tumor, urethral cancer, penile cancer, prostate cancer, chronic or acute leukemia, lymphocytic lymphoma, bladder cancer, kidney cancer, ureter cancer, renal cell carcinoma, renal pelvic carcinoma, CNS tumor, glioma, astrocytoma, glioblastoma multiforme, primary CNS lymphoma, bone marrow tumor, brain stem nerve gliomas, pituitary adenoma, uveal melanoma (also known as intraocular melanoma), testicular cancer, oral cancer, pharyngeal cancer or a combination thereof.

Disclosed herein is a method of treating a subject with a PLK1 inhibiting therapy, the method comprising; a) determining the level and/or activity of AT-rich interacting domain 1A (ARID1A) in a sample obtained from the subject to predict the responsiveness of the subject towards the PLK1 inhibiting therapy; and b) treating a subject found likely to be sensitive towards the PLK1 inhibiting therapy.

In one embodiment, there is provided a method of treating a subject with a PLK1 inhibiting therapy, the method comprising administering a PLK1 inhibiting therapy to the subject, wherein the subject is found to have a cancer having reduced level and/or activity of ARID1A, wherein the reduced level and/or activity of ARID1A predicts that the subject is responsive towards the PLK1 inhibiting therapy.

As used herein, the term “subject” includes any human or non-human animal. In one embodiment, the subject is a human. The term “non-human animal” includes all vertebrates, e.g., mammals and non-mammals, such as non-human primates, sheep, dog, cow, chickens, amphibians, reptiles, etc.

The term “treating” as used herein may refer to (1) preventing or delaying the appearance of one or more symptoms of the disorder; (2) inhibiting the development of the disorder or one or more symptoms of the disorder; (3) relieving the disorder, i.e., causing regression of the disorder or at least one or more symptoms of the disorder; and/or (4) causing a decrease in the severity of one or more symptoms of the disorder.

By “effective amount”, in the context of treating or preventing a condition is meant the administration of an amount of an agent or composition to an individual in need of such treatment or prophylaxis, either in a single dose or as part of a series, that is effective for the prevention of incurring a symptom, holding in check such symptoms, and/or treating existing symptoms, of that condition. The effective amount will vary depending upon the health and physical condition of the individual to be treated, the taxonomic group of individual to be treated, the formulation of the composition, the assessment of the medical situation, and other relevant factors. It is expected that the amount will fall in a relatively broad range that can be determined through routine trials.

In one embodiment, there is provided a method of treating a subject with a PLK1 inhibiting therapy, the method comprising (a) selecting a subject who is predicted to be responsive towards the PLK1 inhibiting therapy, and (b) treating the subject with the PLK1 inhibiting therapy. The subject may be one who is found to have a decreased or reduced level and/or activity of ARID1A in a sample obtained from the subject as compared to a reference. This indicates that the subject is likely to be responsive to the PLK1 inhibiting therapy.

Disclosed herein is a method of stratifying a subject as a likely responder or non-responder towards PLK1 inhibiting therapy, the method comprising determining the level and/or activity of AT-rich interacting domain 1A (ARID1A) in a sample obtained from the subject to predict the responsiveness of the subject towards the PLK1 inhibiting therapy, to thereby stratify the subject as a likely responder or non-responder towards the PLK1 inhibiting therapy.

As used herein, the terms “stratifying” and “classifying” are used interchangeably herein to refer to sorting of subjects into different strata or classes based on the features of a particular physiological or pathophysiological state or condition. For example, stratifying a population of subjects according to whether they are likely to respond to a PLK1 inhibiting therapy involves assigning the subjects based on the level and/or activity of AT-rich interacting domain 1A (ARID1A) in a sample obtained from the subject.

Disclosed herein is a method of predicting the treatment outcome of a subject towards a PLK1 inhibiting therapy, the method comprising determining the level and/or activity of AT-rich interacting domain 1A (ARID1A) in a sample obtained from the subject to predict the responsiveness of the subject towards the PLK1 inhibiting therapy; and thereby predict the treatment outcome of the subject towards the PLK1 inhibiting therapy.

As used herein, “treatment outcome” refers to predicting the response of a cancer patient to a selected therapy or treatment, including the likelihood that a patient will experience a positive or negative outcome with a particular treatment. As used herein, “indicative of a positive treatment outcome” or the like refers to an increased likelihood that the patient will experience beneficial results from the selected treatment (e.g., complete or partial response, complete or partial remission, reduced tumor size, stable disease, etc.). By contrast, “indicative of a negative treatment outcome” or the like is intended to mean an increased likelihood that the patient will not benefit from the selected treatment with respect to the progression of the underlying cancer (e.g., progressive disease, disease recurrence, increased tumor size, etc.).

Provided herein are also compositions for predicting the responsiveness towards a Polo-like kinase 1 (PLK1) inhibiting therapy in a subject. The composition may comprise an antibody that binds specifically to ARID1A and a sample obtained from the subject. The sample may comprise one or more cancer cells. The antibody may optionally be conjugated to a detectable label such as a fluorophore. The composition may further comprise an antibody that binds to a tumor biomarker (such as an epithelial biomarker).

Provided herein is also a kit for predicting the responsiveness towards a Polo-like kinase 1 (PLK1) inhibiting therapy in a subject. The kit may comprise an antibody that binds specifically to ARID1A. The kit may comprise a suitable buffer and other components for isolating and/or processing a sample from the subject. The kit may further comprise an antibody that binds to a tumor biomarker (such as an epithelial biomarker).

By “about” is meant a quantity, level, value, number, frequency, percentage, dimension, size, amount, weight or length that varies by as much 15, 14, 13, 12, 11, 10, 9, 8, 7, 6, 5, 4, 3, 2 or 1% to a reference quantity, level, value, number, frequency, percentage, dimension, size, amount, weight or length.

As used herein, “and/or” refers to and encompasses any and all possible combinations of one or more of the associated listed items, as well as the lack of combinations when interpreted in the alternative (or).

As used in this application, the singular form “a” “an” and “the” include plural references unless the context clearly dictates otherwise. For example, the term “an agent” includes a plurality of agents, including mixtures thereof.

Throughout this specification and the statements which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

Those skilled in the art will appreciate that the invention described herein in susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

### Examples

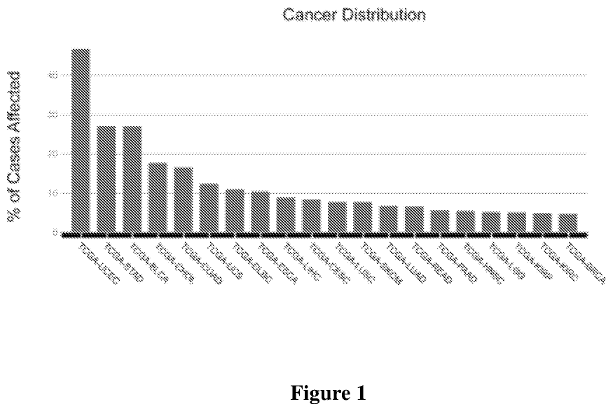

The loss of ARID1A was identified to predict the sensitivity to PLK1 inhibition. ARID1A is a tumor suppressor with high mutational rates ˜20% in all cancers and is therefore a target for synthetic lethal studies (FIG. 1).

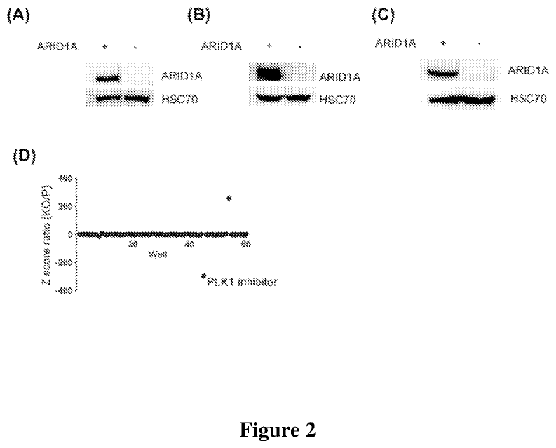

ARID1A Knockout (KO) cell lines were generated using CRISPR-Cas9 gene editing in multiple cellular contexts where ARID1A is lost-Gastric (GES1), Breast (MCF10A), and Ovarian (OVCAR3) (FIG. 2 A-C). A cell survival screen in the MCF10A system with clinically applicable kinase inhibitors identified that cells with a loss of ARID1A had preferentially sensitivity to a PLK1 inhibitor (TAK960) (FIG. 2 D).

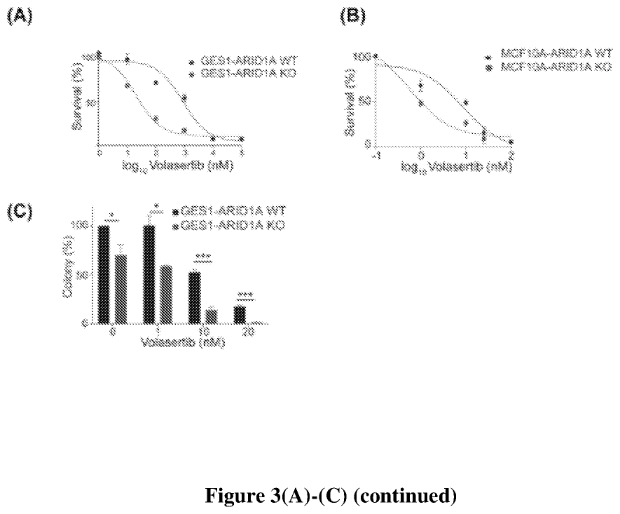

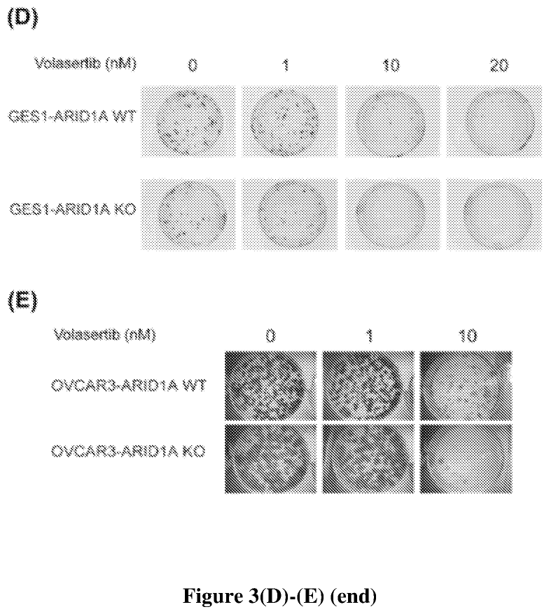

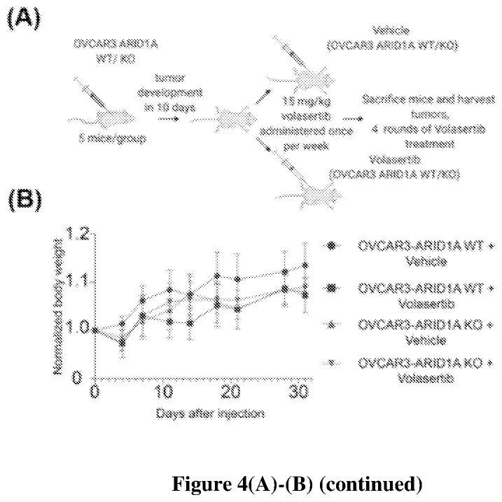

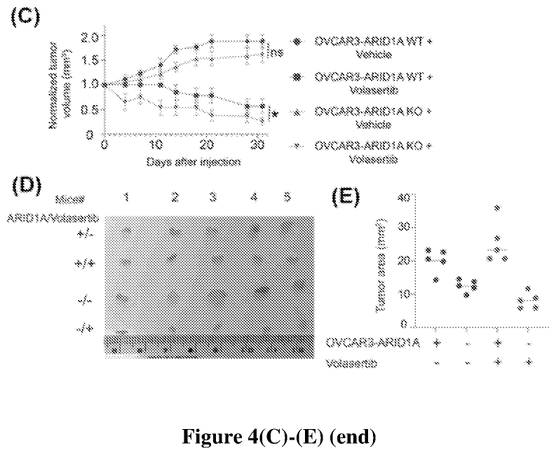

This synthetic lethal interaction was validated using Volasertib in multiple isogenic ARID1A WT and KO cell lines, using cell proliferation and colony formation assays (FIG. 3 A-E). This synthetically lethal relationship was also demonstrated in an in vivo mouse xenograft mouse model (FIG. 4A-E).

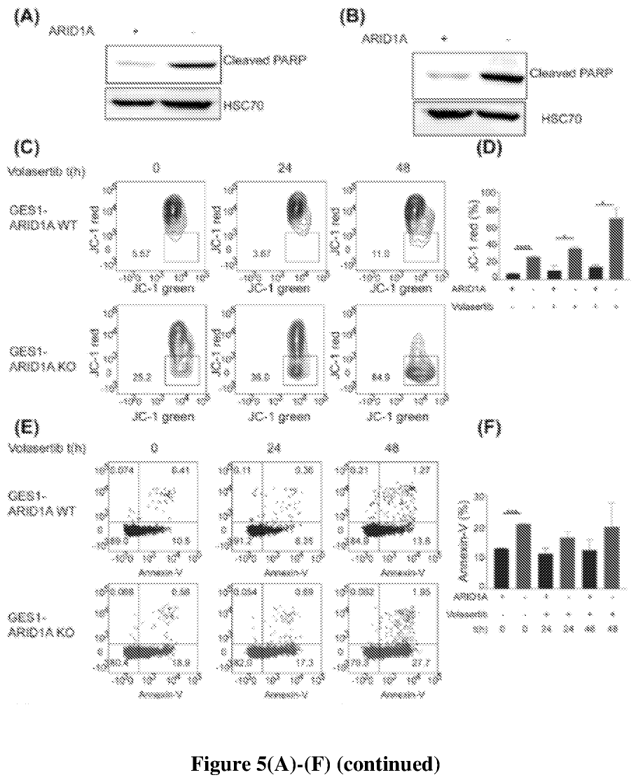

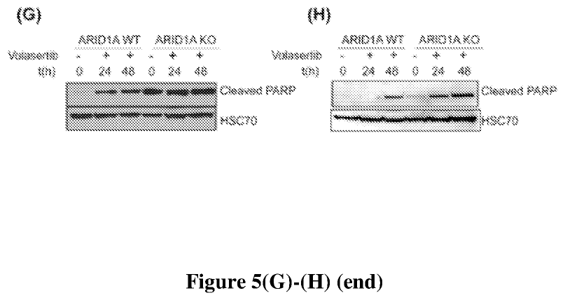

ARID1A mutant cells were seen to have a high baseline level of apoptosis due to baseline mitochondrial depolarization, and that Volasertib treatment markedly increases mitochondrial depolarization; leading to apoptosis and selective cell killing in ARID1A KO cells (FIG. 5 A-H).

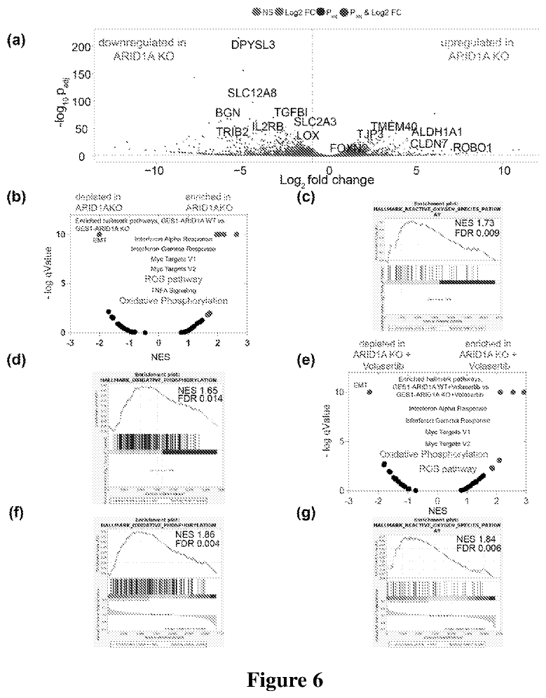

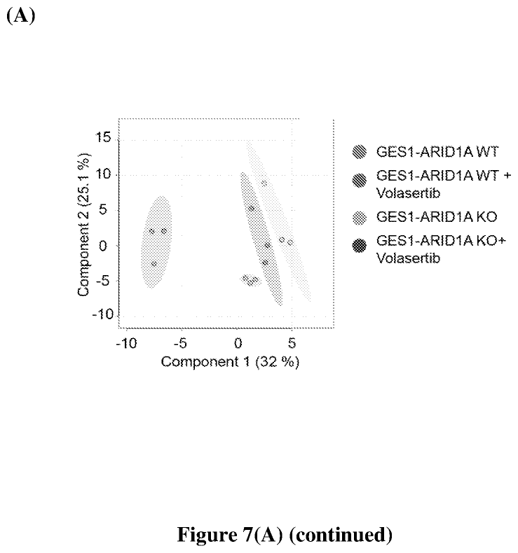

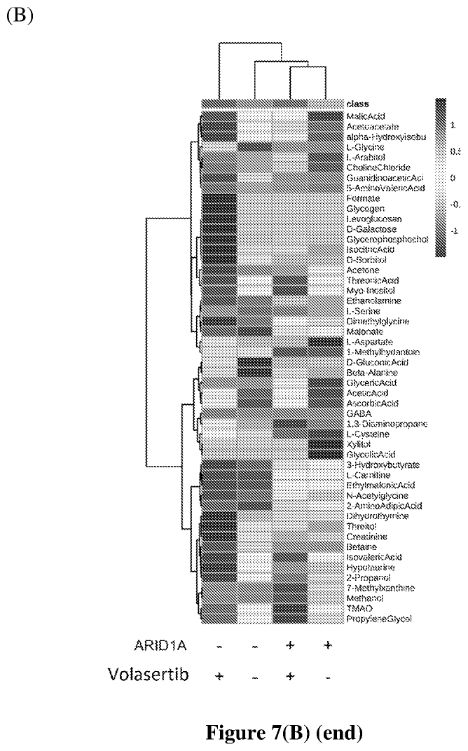

In addition, ARID1A loss causes a dramatic alteration in transcriptome leading to up or down regulation of many important biological pathways (FIG. 6). The effect of ARID1A loss on metabolomics was also confirmed using 1D NMR, with volasertib treatment causing profound changes in the metabolomics (FIG. 7 A-B).

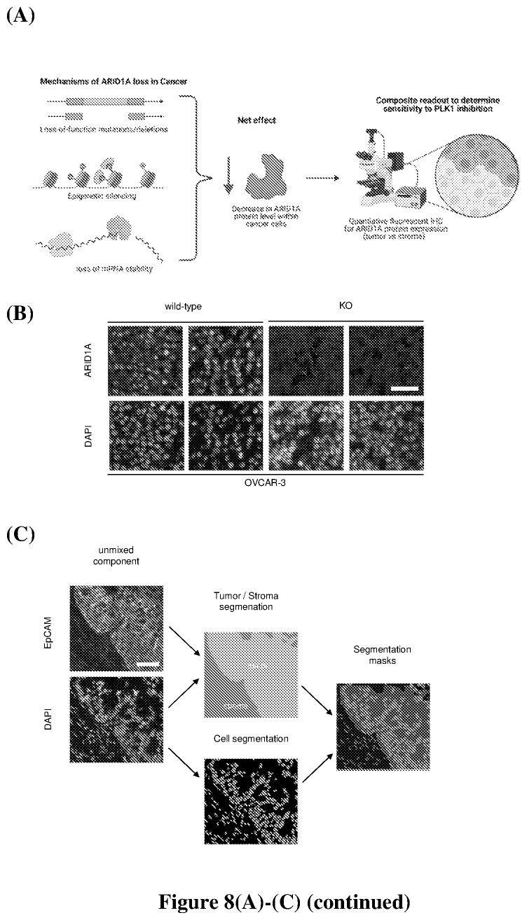

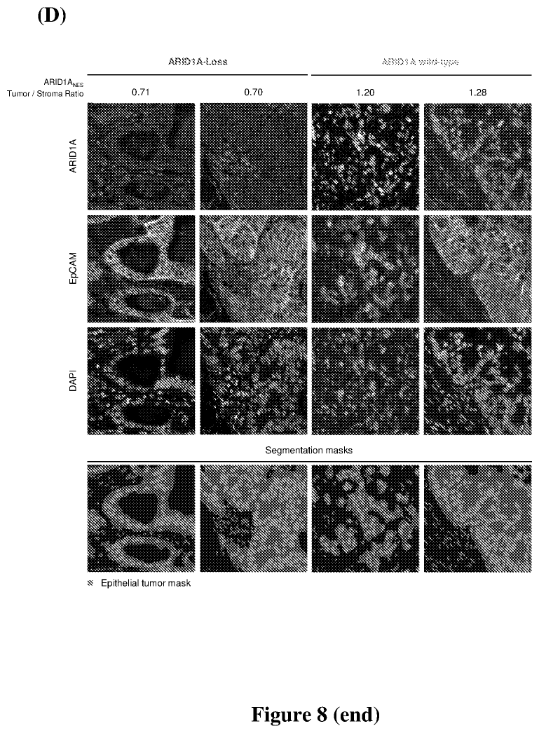

The study provides a rationale for the use of PLK1 inhibitors in tumors with ARID1A loss. Using this as a premise, a quantitative immunohistochemistry (qIHC) assay has been developed and optimized for measurement of ARID1A expression in tumors (FIG. 8A). As there are multiple ways in which ARID1A loss can occur (including loss of function mutations, epigenetic suppression and aberrant mRNA processing), the measurement of ARID1A at the protein level is the most comprehensive way to assess ARID1A loss (FIG. 8A). A rabbit monoclonal antibody (ab182561) was validated for reliable detection of ARID1A expression in FFPE samples. The antibody recognized a clear nuclear signal in FFPE cell-blocks (consistent with ARID1A function as chromatin remodeler), which was abrogated in knock-out cell blocks (FIG. 8B). The Vectra 2 imaging system was used to quantify ARID1A expression in patient samples. Briefly, once qIHC staining for ARID1A is performed, images are taken by the Vectra microscope. The Vectra system software is ‘taught’ to first distinguish between stromal and tumor cells. Subsequently the tissue is segmented into single cells. For each denoted cell, the mean fluorescent intensity measurement for ARID1A staining is captured. As ARID1A is predominately expressed in the nucleus, an ARID1A nuclear expression score (ARID1A_{NES}) was initially established as an overall assessment of ARID1A nuclear protein expression per patient case, which is reflective of the average intensity of ARID1A nuclear protein across all imaged cells. As ARID1A loss is frequently observed in tumor cells, but not stromal cells, a marker of epithelial cells was used to differentiate the quantitative analysis between tumor and stromal cells (FIG. 8C). A Tumor/Stroma ARID1A_{NES} Ratio was therefore used as a readout for ARID1A loss (FIG. 8D). This was taken to be the mean nuclear intensity of ARID1A within tumour cells divided by mean nuclear intensity of ARID1A within stromal cells. In this assessment, gastric cancer cases with a visually appreciable loss of ARID1A protein expression show a Tumor/Stroma ARID1A_{NES} Ratio <1 (FIG. 8D). This method could be widely applied across cancer types for the identification of cases with loss of ARID1A expression in tumor cells, and therefore likelihood of response to PLK1 inhibition.

1. A method for predicting the responsiveness of a subject towards a Polo-like kinase 1 (PLK1) inhibiting therapy, the method comprising determining the level and/or activity of AT-rich interacting domain 1A (ARID1A) in a sample obtained from the subject.

2. A method for predicting the responsiveness of a subject towards a Polo-like kinase 1 (PLK1) inhibiting therapy, the method comprising determining the mutation status of AT-rich interacting domain 1A (ARID1A) in a sample obtained from the subject.

3. A method for predicting the responsiveness of a subject towards a Polo-like kinase 1 (PLK1) inhibiting therapy, the method comprising determining transcriptomic and/or metabolic changes due to AT-rich interacting domain 1A (ARID1A) mutation(s) or loss in a sample obtained from the subject.

4. The method of claim 1, wherein an increased level and/or activity of ARID1A as compared to a reference indicates a likelihood of resistance towards the PLK1 inhibiting therapy.

5. The method of claim 1, wherein a decreased level and/or activity of ARID1A as compared to a reference indicates a likelihood of sensitivity towards the PLK1 inhibiting therapy.

6. The method of claim 1, wherein the PLK1 inhibiting therapy comprises a PLK1 inhibitor.

7. The method of claim 2, wherein the subject is suffering from cancer.

8. The method of claim 7, wherein the cancer is a gastric or ovarian cancer.

9. The method of claim 1, wherein the sample comprises one or more cancer cells.

10. The method of claim 1, wherein the method further comprises detecting a tumor biomarker in the one or more cancer cells.

11. The method of claim 1, wherein the method comprises administering an inhibitor that is synthetically lethal with ARID1A loss to the subject.

12.-14. (canceled)
